(19) United States
(12) Patent Application Publication
(10) Pub. No.: US 2021/0007944 A1
(43) Pub. Date: Jan. 14, 2021

(54) SOLID COSMETIC DETERGENTS (TWO-COMPONENT COMPOSITION)

(71) Applicant: Henkel AG & Co. KGaA, Duesseldorf (DE)

(72) Inventors: SOEREN SCHEELE, Pinneberg (DE); MANUELA METTE, Kleinfeld (DE); PETRA WESTPHAL, Neu Wulmstorf (DE); THOMAS SCHROEDER, Hamburg (DE)

(73) Assignee: Henkel AG & Co. KGaA, Duesseldorf (DE)

(21) Appl. No.: 16/926,069

(22) Filed: Jul. 10, 2020

(30) Foreign Application Priority Data
Jul. 10, 2019 (DE) 10 2019 210 157.7

(51) Int. Cl. A61K 8/02; A61K 8/73; A61K 8/60; A61K 8/34; A61Q 5/12

(52) U.S. Cl. CPC A61K 8/0233; A61K 8/731; A61Q 5/12; A61K 8/60; A61K 8/34; A61K 8/732

(57) ABSTRACT

A solid cosmetic composition comprises at least two separate Components I. and II., wherein said at least one Component I comprises at least one of starch fractions from corn, potatoes, rice, wheat and/or tapioca, cereal flour and/or sugar chosen from glucose, sucrose, fructose, maltose, and said at least one Component II comprises from about 15.0 to about 50.0% by weight of at least one surfactant, from about 10.0 to about 60.0% by weight of at least one polyhydric C₂-C₆ alcohol, from about 0.1 to about 10.0% by weight of at least one saturated or unsaturated, branched or unbranched C₈-C₃₀-alcohol and/or a saturated or unsaturated alcohol, branched or unbranched C₈-C₃₀carboxylic acid and/or a salt of a saturated or unsaturated, branched or unbranched C₈-C₃₀carboxylic acid, and from about 0.1 to about 20.0% by weight of at least one polysaccharide.

### CROSS-REFERENCE TO RELATED APPLICATION

This application claims priority to German Patent Application No. 10 2019 210 157.7, filed Jul. 10, 2019, which is incorporated herein by reference in its entirety.

### TECHNICAL FIELD

The notification describes solid cosmetic compositions based on surfactants, specific polyols, polysaccharides, fatty alcohols and/or fatty acid(s), in particular solid cleansing compositions which dissolve and foam in contact with water. The notification also describes processes for the preparation of solid cosmetic compositions and their use for cleansing the human body, including the hair.

### BACKGROUND

Surfactant-comprising products for cleansing the human body and hair have been known for a long time and are mainly sold as foam baths, shower preparations, shampoos and/or oral care compositions in liquid or paste form in suitable packaging. End users take the required amount of product from the packaging during use and dispose of it after emptying. Compared to classic soaps that are sold in solid form, such products offer the user the advantage of easy and quick handling, which is why they dominate the market today. However, this advantage is achieved by accepting certain disadvantages, which are discussed below. In most cases, the packaging of the described products in liquid or paste form includes non-recyclable plastic, which is a serious problem from an environmental point of view in view of the constantly growing amount of plastic waste.

Another problem is that previous products usually comprise higher quantities of water or water/solvent mixtures, which means that the products have a larger volume and, possibly of greater importance from a transport point of view, a relatively high weight. This is disadvantageous for several reasons. In times of increasing water scarcity, resources should be saved. An undesirable, increased transport volume associated with large-volume heavy products is also important from an environmental and cost perspective. Another interesting point is that worldwide travel activity is constantly increasing. Consumers are therefore increasingly interested in cosmetic products that are easy to transport due to their low weight and volume. This is particularly relevant with regard to air travel, as larger containers comprising liquids are generally excluded from being carried in an aircraft cabin, so that a passenger travelling only with hand luggage often finds himself in the situation, due to the cosmetics products that dominate the market today, of not being able to take his typical product selection with him or having to decant the corresponding products into smaller containers first, which, however, is generally accompanied by an even greater volume of packaging material.

The provision of alternative product forms with lower water content, which are included in more environmentally friendly, for example recyclable, packaging to save space, is therefore an important goal in the formulation of improved, contemporary and sustainable cosmetic products.

Classic bar soaps have long been known and used. They also generally have a very low water or solvent content and are often packed with little material but their handling is not pleasant for many people because a started soap bar is difficult to transport, often partially dissolves when lying next to the sink, which is also inefficient, and makes the sink look unattractive due to soap residues and because soap bars have a tendency to slip out of the user's hand when wet.

Furthermore, classic soap formulations are only conditionally suitable for the regular cleaning of the entire human body. Due to their skin dehydration potential, they are mainly used for cleaning hands, which are more tolerant in this respect than other skin areas. Furthermore, bar soaps are controversial from a hygiene point of view because of their multiple uses, in particular when they are used by several final consumers.

Another disadvantage of conventional soap formulation, especially with rather small bars of soap, is that it takes some time for enough of the soap bar to come off to achieve the desired amount of lather and the desired cleaning effect. On the one hand, this is usually not desired by users because of the additional time required, and on the other hand it can be associated with higher water consumption for personal hygiene, as many users do not turn off the water flow of the shower or tap when soaping. From these points of view, it does not make sense to market single application portions of classic soap formulations, especially in miniaturized form of a classic soap bar, as their dissolution is too slow as the formulations of these soaps are not optimized for cleaning larger skin areas or hair.

### BRIEF SUMMARY

In view of the problems and requirements described above, formulations and manufacturing processes suitable for solid cosmetic detergents are provided which, by their nature, can be packaged in individual application portions, as well as processes for their manufacture and uses. Several measures that can contribute to this suitability. Thus, the task of the present disclosure is solved by the formulation of procedures and uses described in detail below:

The present disclosure provides:
1. A solid cosmetic composition comprising two separate Components I. and II., wherein
comprising at least one Component I.
a. Starch fractions from corn, potatoes, rice, wheat and/or tapioca,
b. cereal flour and/or
c. sugar selected from the group including glucose, sucrose, fructose, maltose, and comprising at least one Component II.
a. from about 15.0 to about 50.0% by weight of at least one surfactant,
b. from about 10.0 to about 60.0% by weight of at least one polyhydric C₂-C₆ alcohol
c. from about 0.1 to about 10.0% by weight of at least one saturated or unsaturated, branched

or unbranched C₈-C₃₀-alcohol and/or a saturated or unsaturated alcohol, branched or unbranched C₈-C₃₀carboxylic acid and/or a salt of a saturated or unsaturated, branched or unbranched C₈-C₃₀ carboxylic acid, and
d. From about 0.1 to about 20.0% by weight of at least one polysaccharide selected from
Starch fractions, modified starches, derivatives of starches,
cellulose and/or cellulose derivatives
where the quantities stated refer to the total weight of Component II.
2. A solid cosmetic composition according to point 1, comprising in Component I.—based on its total weight from about 75 to about 100% by weight of one or more ingredient(s) a. to c.
3. A solid cosmetic composition according to any of the preceding points, comprising in Component I.—based on its total weight—from about 75 to about 100% by weight of one or more ingredient(s) from Group a.
4. A solid cosmetic composition according to any of the preceding points, comprising in Component I.—based on its total weight—from about 75 to about 100% by weight of potato starch.
5. A solid cosmetic composition according to any of the preceding points, comprising in Component I.—based on its total weight—
a. from about 0 to about 10% by weight of native vegetable oils and/or
b. from about 0 to about 15% by weight of water.
6. A solid cosmetic composition according to any of the preceding points, comprising as surfactant a) in Component II. at least one anionic surfactant.
7. A solid cosmetic composition according to any of the preceding points, comprising in Component II.—based on the total weight of Component II.—from about 17.5 to about 47.5% by weight, typically from about 20.0 to about 45.0% by weight, particularly typically from about 22.5 to about 42.5% by weight and in particular from about 25.0 to about 40.0% by weight of at least one surfactant a), typically an anionic surfactant.
8. A solid cosmetic composition according to any of the preceding points, comprising as surfactant a) in Component II.
i. at least one anionic isethionate surfactant of formula (I)

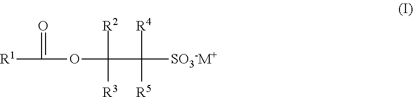

located in the
R¹ represents a linear or branched, saturated or unsaturated alkyl radical comprising about 6 to about 30 carbon atoms,
R² to R⁵ independently of one another represent a hydrogen atom, a C₁-C₄ alkyl radical or a C₁-C₄₄ hydroxyalkyl radical, and/or
M+ represents an ammonium, an alkanolammonium or a metal cation, and/or p0 ii. at least one anionic taurate surfactant of formula (II)

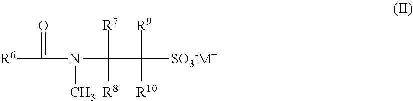

1.
located in the
   R⁶ represents a linear or branched, saturated or unsaturated alkyl radical comprising about 6 to about 30 carbon atoms,
R⁷ to R¹⁰ independently of one another represent a hydrogen atom, a C₁-C₄ alkyl radical or a C₁-C₄ hydroxyalkyl radical, and

M+ stands for an ammonium, an alkanol ammonium or a metal cation.
9. A solid cosmetic composition according to point 8, comprising in Component II. at least one anionic isethionate surfactant according to formula (I) and at least one anionic taurate surfactant according to formula (II).
10. A solid cosmetic composition according to one of points 8 or 9, comprising (based on the total weight of Component II.).
i. From about 10.0 to about 40% by weight, typically from about 12.5 to about 35% by weight, particularly typically from about 15.0 to about 32.5% by weight and in particular from about 17.5 to about 30.0% by weight of at least one anionic An isethionate surfactant of formula (I) and
ii. From about 1.0 to about 10% by weight, typically from about 2.0 to about 9.0% by weight, particularly typically from about 2.5 to about 8.0% by weight and in particular from about 3.0 to about 7.0% by weight of at least one anionic taurate surfactant corresponding to formula (II).
11. A solid cosmetic composition according to any of the preceding points, comprising in Component II. at least one polyhydric C2-C6 alcohol b) in an amount of from 15.0 to 60% by weight, typically from about 20.0 to about 60% by weight, particularly typically from about 25.0 to about 60% by weight and more particularly from about 30.0 to about 60% by weight (based on the total weight of Component II.).
12. A solid cosmetic composition according to any of the preceding points, comprising as polyhydric alcohol b) in component II. from about 1.2-propylene glycol, about 1.3-butylene glycol, dipropylene glycol, glycerol and/or diglycerol, typically glycerol.
13. A solid cosmetic composition according to one of points 11 or 12, comprising as polyhydric alcohol b) in Component II Glycerine.
14. A solid cosmetic composition according to any of the preceding points, comprising in Component II. at least one Component c) in an amount of from about 0.5 to about 10% by weight, typically from about 1.0 to about 10% by weight, more typically from about 2.0 to about 10% by weight and more typically from about 3.0 to about 10% by weight (based on the total weight of Component II.).
15. A solid cosmetic composition according to one of the preceding points, comprising as Component c) in Component II. saturated or unsaturated, branched or unbranched C₈-C₃₀-carboxylic acids and/or their salts, typically C₁₀-C₂₂-carboxylic acids and/or their salts and in particular coco acids, lauric acid, myristic acid, palmitic acid, stearic acid, behenic acid, oleic acid and mixtures thereof and/or the salts of these acids.
16. A solid cosmetic composition according to one of points 14 or 15, comprising in Component II. Coco acids, stearic acid and/or the salts of these acids, in particular coco acids and sodium stearates.
17. A solid cosmetic composition according to any of the preceding points, comprising in Component II. at least one Component d) in an amount of from about 0.5 to about 17.5% by weight, typically from about 1.0 to about 15.0% by weight, more typically from about 1.5 to about 12.5% by weight and more typically from about 2.0 to about 10.0% by weight (based on the total weight of Component II.).
18. A solid cosmetic composition according to any of the preceding points, comprising as polysaccharide d) in Component II. as polysaccharide d)
i. Starch fractions from maize, potatoes, rice, wheat and/or tapioca and/or derivatives of starches such as amylose, amylopectin, dextrins and/or
ii. Cellulose and/or cellulose derivatives such as alkyl cellulose, carboxyalkyl cellulose, hydroxyalkyl cellulose.
19. A solid cosmetic composition according to any of the preceding points, comprising as at least one polysaccharide d) in Component II. one or more ingredients from group.
20. A solid cosmetic composition according to point 19, comprising as polysaccharide d) in Component II. (based on the total weight of Component II.)
   from about 1.0 to about 15% by weight of maize starch and
   from about 0.1 to about 10% by weight of dextrins, typically maltodextrin.
21. A solid cosmetic composition according to any of the preceding points, comprising in Component II. at least one cationic polymer, typically at least one cationic polysaccharide polymer obtainable from guar, cassia and/or inulin in a proportion by weight of from about 0.01 to about 5.00% by weight of the total weight of Component II.
22. A solid cosmetic composition according to item 21, comprising at least one cationic polymer known under the INCI name hydroxypropyl trimonium inulin.
23. A solid cosmetic composition according to one of the preceding points, comprising in Component II. At least one oil, fat and / or wax component, typically a naturally occurring oil, fat or wax in a weight fraction of from about 0.01 to about 5.00 parts by weight. % of the total weight of Component II.
24. A solid cosmetic composition according to item 23, comprising apricot kernel oil, argan oil, jojoba oil, marula oil, almond oil, olive oil, coconut oil and/or sunflower oil.
25. A solid cosmetic composition according to one of the preceding points, comprising in Component I and/or II at least one bitter substance, typically a compound known under the INCI designation denatonium benzoate in a proportion by weight of from about 0.001 to about 0.01% by weight of the total weight of Component I and/or II.
26. A solid cosmetic composition according to any of the preceding points for the cleansing and/or care of human skin and/or human hair.
27. A solid cosmetic composition according to any of the preceding points comprising in Component II. Water in an amount of up to 15% by weight, typically from about 12.5% by weight and in particular about 10% by weight (based on the weight of Component II.).
28. A solid cosmetic composition according to any of the foregoing, wherein
   Component I. was coated with Component II., or
   Component I. has been impregnated, sprayed or coated with Component II., or
   Component II. has been rolled onto Component I., or
   Component I. with Component II. was printed.
29. A solid cosmetic composition according to any of the foregoing, with Component II. being a separate layer between two Components I. (sandwich arrangement).
30. A solid cosmetic composition according to any of the foregoing, comprising an additional layer III comprising a hair conditioning agent.
31. A solid cosmetic composition according to any of points 1 to 27, where Component II. is completely enveloped by Component I.
32. A solid cosmetic composition according to a point 1 to 27 and 29 to 31, wherein Component II. is in the form of a porous body which has a density in the range from about 0.2 g/cm³ to about 1.2 g/cm³ and which dissolves and foams in contact with water.
33. A method for cleansing the human body including the hair, in which a solid cosmetic composition is moistened with water according to any one of points 1 to 32, soaked with water or dissolved in water, rubbed between the hands and foamed, spread over the part of the body to be cleansed and rinsed with water.
34. A method of applying a solid cosmetic composition according to one of points 1 to 32, wherein
a) the solid composition is first mixed with water and then applied to the hair and worked in, or
b) the solid composition is applied directly to the wet hair and worked in.
35. The use of a cleansing composition according to any of points 1 to 32 for the cleansing and/or care of the human body including the hair.

In one embodiment, a solid cosmetic composition comprises at least two separate Components I. and II., wherein said at least one Component I comprises at least one of: starch fractions from corn, potatoes, rice, wheat and/or tapioca, cereal flour and/or sugar chosen from glucose, sucrose, fructose, maltose, and said at least one Component II comprises: from about 15.0 to about 50.0% by weight of at least one surfactant, from about 10.0 to about 60.0% by weight of at least one polyhydric C₂-C₆ alcohol, from about 0.1 to about 10.0% by weight of at least one saturated or unsaturated, branched or unbranched C₈-C₃₀-alcohol and/or a saturated or unsaturated alcohol, branched or unbranched C₈-C₃₀ carboxylic acid and/or a salt of a saturated or unsaturated, branched or unbranched C₈-C₃₀ carboxylic acid, and from about 0.1 to about 20.0% by weight of at least one polysaccharide chosen from starch fractions, modified starches, derivatives of starches, and cellulose and/or cellulose derivatives, wherein the quantities are based on the total weight of said at least one Component II.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

A first subject of this notification is a solid cosmetic composition comprising two separate Components I. and II.
comprising at least one Component I.
   a. Starch fractions from corn, potatoes, rice, wheat and/or tapioca,
   b. cereal flour and/or
   c. comprises sugar chosen from glucose, sucrose fructose, maltose, and
comprising at least one Component II.
   a. from about 15.0 to about 50.0% by weight of at least one surfactant,
   b. from about10.0 to about 60.0% by weight of at least one polyhydric C₂-C₆ alcohol
   c. from about 0.1 to about 10.0% by weight of at least one saturated or unsaturated, branched or unbranched C₈-C₃₀alcohol and/or a saturated or unsaturated, branched or unbranched C₈-C₃₀carboxylic acid and/or a salt of a saturated or unsaturated, branched or unbranched C₈-C₃₀carboxylic acid, and
   d. from about 0.1 to about 20.0% by weight of at least one polysaccharide selected from starch fractions, modified starches, derivatives of starches, cellulose and/or cellulose derivatives
   where the quantities stated refer to the total weight of Component II.

The compositions as contemplated herein are solid at about 25° C. Solid compositions within the meaning of the present application are three-dimensional, dimensionally stable entities which are not liquid or gaseous, that is to say, which retain their external shape even without a surrounding vessel. However, the term “solid” does not imply anything about density or elasticity or other physical properties, so that jellies, brawn, butter etc. can also be solid as contemplated herein as long as they are dimensionally stable at about 25° C.

Component I. serves here, for example, as a coating, matrix and/or base (in the form of a thin flake and/or wafer) for Component II. This creates more freedom in the formulation of Component II, as its strength and surface properties are less restricted. The ingredients listed for Component I. come from natural raw materials, are therefore sustainably usable, harmless to humans and the environment and are well suited for this application due to their structure-giving properties.

Component I. may comprise cosmetic active ingredients for skin and/or hair care. However, this is not absolutely necessary as typically the active substances required for skin and/or hair care are included in Component II. of the solid compositions as contemplated herein.

Component II represents the primarily cosmetically effective composition.

A formulation as just described offers the right properties for single application portions, especially with regard to its dissolving and foaming behaviour during use. The high concentrations of the active substances in such a composition are associated with the advantages that few resources are consumed during production and transport and that the products, even after they have reached the hands of a consumer via the trade, can be easily transported without great effort or restrictions, whether to the gym or on a flight.

### Description of Component I:

The present disclosure further relates to a solid cosmetic composition as described above, comprising in Component I.—based on its total weight—from about 75 to about 100% by weight of one or more ingredient(s) from groups a. to c., typically from about 75 to about 100% by weight of an ingredient from group a. and in particular from about 75 to about 100% by weight of potato starch.

The mentioned polysaccharides (I.a.), in particular potato starch, have proved to be well suited as stabilising agents in the context of the present disclosure claimed here. Their use makes it possible to provide ready-made consumer products that retain their properties and appearance over a long period of time and under various environmental conditions. These advantages are particularly pronounced when implementing the concentration ranges mentioned.

The present disclosure further relates to a solid cosmetic composition as described above, comprising in Component I.—based on its total weight—
from about 0 to about 10% by weight of native vegetable oils and/or
from about 0 to about 15% by weight of water.

The solid cosmetic compositions as described above typically comprise in Component I.—based on the total weight of Component I.—
from about 0 to about 8, more typically from about 0 to about 6% by weight, more typically from about 0 to about 4% by weight and more typically from about 0 to about 2% by weight of native vegetable oils and/or
from about 0 to about 15% by weight, more typically from about 0 to about 14% by weight, more typically from about 0 to about 12.5% by weight and more typically from about 0 to about 10% by weight of water.

Specific suitable oils will be discussed later. Naturally occurring raw materials have the advantage that they grow again and can therefore be used sustainably. This aspect is also becoming increasingly important to many users. In addition, some vegetable oils or butters, especially if they have been carefully extracted at low temperatures, are extremely potent care products for skin and hair, as they also comprise a large number of certain secondary ingredients such as vitamins A certain water content may be advantageous to adjust the properties of the finished solid cosmetic composition and/or to facilitate its preparation.

### Description of Component II.:

The solid cosmetic composition described above may comprise as at least one surfactant a) Surfactant(s) from the group of anionic, amphoteric, zwitterionic or non-ionic surfactants or a mixture thereof.

In a typical embodiment, the compositions as contemplated herein comprise—based on the total weight of Component II—from about 17.5 to about 47.5% by weight, typically from about 20.0 to about 45.0% by weight, particularly typically from about 22.5 to about 42.5% by weight and in particular from about 25.0 to about 40.0% by weight of at least one anionic surfactant a).

Anionic surfactants carry a negative charge in their hydrophilic part. Chemically, these are usually salts of sodium, potassium, ammonium, magnesium, monoethanolamine, diethanolamine or triethanolamine

Among the basically suitable anionic surfactant types which can be used in the agents as contemplated herein are, for example:
linear and branched fatty acids with about 8 to about 30 C atoms (Soaps),
Ethercarboxylic acids of the formula R—O—(CH₂—CH₂O)ₓ—CH₂—COOH, in which R is a linear or branched, saturated or unsaturated alkyl group having about 8 to about 30 C atoms and x=from about 0 or from about 1 to about 16.
Acylsarcosides with about 8 to about 24 C atoms in the acyl group (sarcosinate surfactants)
Acyltaurides with about 8 to about 24 C atoms in the acyl group (taurate surfactants),
Acyl isethionates with about 8 to about 24 C atoms in the acyl group (isethionate surfactants),
Sulphosuccinic acid mono- and/or dialkyl esters with about 8 to about 24 C atoms in the alkyl group and Sulphosuccinic acid mono-alkyl polyoxyethyl esters with about 8 to about 24 C atoms in the alkyl group and 1 to 6 oxyethyl groups (sulphosuccinate surfactants),
Alpha-olefin sulphonates with about 8 to about 24 C atoms (alpha-olefin sulphonates surfactants),
Alkyl sulphates and/or alkyl ether sulphate salts of the formula R—(OCH₂—CH₂)ₙ—O—SO₃X, in which R is typically a straight-chain or branched, saturated or unsaturated alkyl group comprising about 8 to about 30 carbon atoms, x is the number 0 or 1 to 12 and X is an alkali metal, alkaline earth metal, ammonium or alkanolamine ion,
Sulphonates of unsaturated fatty acids with about 8 to about 24 C atoms and 1 to 6 double bonds,
Esters of tartaric acid and citric acid with alcohols which are addition products of about 2-15 molecules of ethylene oxide and/or propylene oxide to fatty alcohols with about 8 to about 22 C atoms, and/or
Alkyl and/or alkenyl ether phosphates of the formula

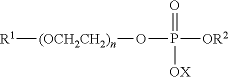

in which R¹ is typically an aliphatic hydrocarbon radical having about 8 to about 30 carbon atoms, R² for hydrogen, a radical (CH₂CH₂O)ₙR¹ or X, n is numbers from 0 to 10 and X is hydrogen, an alkali metal or alkaline earth metal or the group —NR³R⁴R⁵R⁶ where R³ to R⁶ independently of one another are a C₁ bis C₄ hydrocarbon radical.

Amino acid-based surfactants are particularly suitable in the sense of the present disclosure because they exhibit excellent skin compatibility and they are available from natural sources.

Due to their particularly good incorporation into solid compositions within the meaning of the present disclosure, isethionate and/or taurate surfactants have proved to be particularly suitable.

The present disclosure therefore also concerns a solid cosmetic composition as described above, comprising in Component II.
i. at least one anionic isethionate surfactant of formula (I)

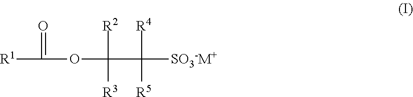

located in the
R¹ represents a linear or branched, saturated or unsaturated alkyl radical comprising about 6 to about 30 carbon atoms,
R² to R⁵ independently of one another represent a hydrogen atom, a C₁-C₄ alkyl radical or a C₁-C₄4 hydroxyalkyl radical, and/or
M+ represents an ammonium, an alkanolammonium or a metal cation and/or
ii. at least one anionic taurate surfactant of formula (II)

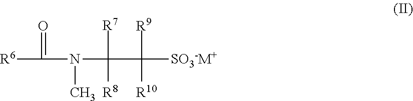

located in the
   R⁶ represents a linear or branched, saturated or unsaturated alkyl radical comprising about 6 to about 30 carbon atoms,
   R⁷ to R¹⁰ independently of one another represent a hydrogen atom, a C₁-C₄ alkyl radical or a C₁-C₄ hydroxyalkyl radical, and
M+ stands for an ammonium, an alkanol ammonium or a metal cation

Anionic isethionate and taurate surfactants are mild, anionic surfactants, ideal for the production of shampoos, shampoo bars, body washes, liquid soaps, face care and bath bombs. They make hair easy to comb, smooth, soft and shiny. They can be produced on a purely vegetable basis and can therefore be used for vegan cosmetics. A raw material source can be fatty acids of coconut oil.

In a particularly typical embodiment, the compositions as contemplated herein comprise in Component II. at least one anionic isethionate surfactant according to formula (I) and at least one anionic taurate surfactant according to formula (II)

The present disclosure also relates to a solid cosmetic composition as described above, comprising in Component II—based on the total weight of Component II.—
i. From about 10.0 to about 40% by weight, typically from about 12.5 to about 35% by weight, particularly typically from about 15.0 to about 32.5% by weight and in particular from about 17.5 to about 30.0% by weight of at least one anionic isethionate surfactant corresponding to formula (I) and
ii. from about 1.0 to about 10% by weight, typically from about 2.0 to about 9.0% by weight, particularly typically from about 2.5 to about 8.0% by weight and in particular from about 3.0 to about 7.0% by weight of at least one anionic taurate surfactant corresponding to formula (II).

These concentration ranges have proven to be particularly suitable. When working with these concentration ranges, the solid cosmetic compositions achievable with them have balanced properties in terms of firmness, solubility behaviour, application feel and cleaning performance

Suitable amphoteric and/or zwitterionic surfactant types which can be used in Component II. of the compositions as contemplated herein include, for example, one or more compounds of the following formulae (i) to (vii) In which the radical R typically represents a straight-chain or branched, saturated or mono- or polyunsaturated alkyl or alkenyl radical having about 7 to about 23 carbon atoms (formulae (i) and (ii)) or a straight-chain or branched, saturated or mono- or polyunsaturated alkyl or alkenyl radical having about 8 to about 24 carbon atoms (formulae (iii) to (vii)):

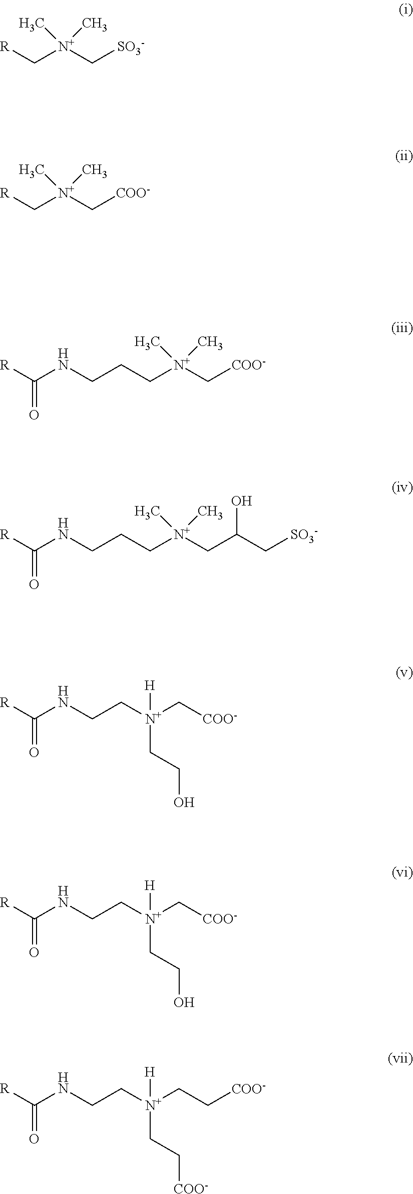

Typical amphoteric surfactants of the formulae (i) to (vii) mentioned above comprise as residue R predominantly a straight-chain or branched, saturated, mono- or polyunsaturated alkyl residue with from about 8 to about 20, more typically from about 8 to about 18 and in particular with about 8 to about 16 C atoms. Particularly typical are amphoteric surfactants, where the rest R is derived from coconut oil.

Particularly typical are those under the INCI designations Sodium Cocoamphoacetate, Disodium Cocoamphoacetate, Sodium Lauroamphoacetate, Sodium Lauroamphoacetate, Sodium Cocoamphopropionate, Disodium Cocoamphodipropionate, Coco Betaine, Lauryl Betaine Cocamidopropylbetaine and/or Lauramidopropylbetaine known and commercially available amphoteric surfactants from several suppliers.

Surfactants with the INCI designations cocamidopropyl betaine, lauramidopropyl betaine, cocoampho(di)acetates and/or lauroapho(di)acetates are particularly typical.

The amphoteric and/or zwitterionic co-surfactant(s) can be used in Component II. of the compositions as contemplated herein (based on the total weight of Component II.) typically in an amount of from about 0 to about 10.0% by weight, more typically from about 0.1 to about 8.0% by weight, particularly typically from about 0.5 to about 6.0% by weight and in particular from about 0.75 to about 5.00% by weight.

Suitable non-ionic surfactants for use as co-surfactants include
Amine oxides, which can be selected from compounds of the general formulas (I) or (II)

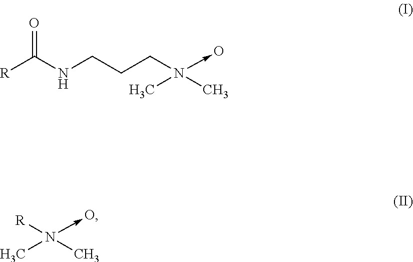

in which each R represents a straight-chain or branched, saturated or mono- or polyunsaturated alkyl or alkenyl radical having about 6 to about 24 carbon atoms, typically about 8 to about 18 carbon atoms.

Particularly typical are the surfactants of the above-mentioned formula (I) or (II) known under the INCI designations Cocamine Oxide, Lauramine Oxide and/or Cocamidopropylamine Oxide and commercially available from various suppliers.
Fatty acid alkanolamides of the following general formula,

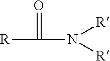

in which R typically denotes a linear or branched, saturated or unsaturated alkyl or alkenyl radical having about 8 to about 24 carbon atoms and the radicals R′ represent hydrogen or the group —(CH₂)ₙOH, in which n denotes the numbers 2 or 3, with the proviso that at least one of the radicals R′ represents the above mentioned radical —(CH₂)ₙOH,
Mixtures of alkyl (oligo)-glucosides and fatty alcohols for example the commercially available product Montanov®68,
Sterols. Sterols are a group of steroids that carry a hydroxyl group on C atom 3 of the steroid skeleton and are isolated from animal tissue (zoosterols) as well as from vegetable fats (phytosterols). Examples of zoosterols are cholesterol and lanosterol. Examples of suitable phytosterols are ergosterol, stigmasterol and sitosterol. Sterols, the so-called mycosterols, are also isolated from fungi and yeasts.
Phospholipids. Among these are mainly the glucose-phospolipids, which are obtained as lecithins or phospahtidylcholines from e.g. egg yolks or plant seeds (e.g. soybeans),
Alkyl(oligo)glycoside. Suitable alkyl (oligo)glycosides can be selected from compounds of the general formula of RO-[G]x, in which [G] is typically derived from aldoses and/or ketosis with 5-6 carbon atoms, typically from glucose.

The index number x stands for the degree of oligomerization (DP), i.e. the distribution of mono- and oligoglycosides. The index number x typically has a value in the range from about 1 to about 10, particularly typically in the range from about 1 to about 3, whereby it may not be an integer but a fractional number that can be determined analytically.

Particularly typical alkyl (oligo)glycosides have a degree of oligomerization between about 1.2 and about 1.5. The radical R typically represents at least one alkyl and/or alkenyl radical comprising about 4 to about 24 carbon atoms.

Especially typical alkyl (oligo)glycosides are the compounds known under the INCI designations Caprylyl/Capryl Glucosides, Decyl Glucosides, Lauryl Glucosides and Coco Glucosides.

Also fatty alcohol alkoxylates (especially ethoxylates) with a C-chain length of about 8 to about 24, especially about 10 to about 20, and degree of alkoxylation (degree of ethoxylation) of about 2 to about 30 such as Laureth-4 etc. are suitable as contemplated herein.

Particularly typical non-ionic surfactants, which may be included as co-surfactants in Component II. of the method as contemplated herein, are fatty acid alkanolamides, alkyl (oligo)glucosides and amine oxides. Especially typical are fatty acid alkanolamides.

The non-ionic co-surfactant(s) can be used in Component II. of the cosmetic compositions as contemplated herein (based on the total weight of Component II.) typically in an amount of from about 0 to about 3.0% by weight, more typically from about 0.1 to about 2.50% by weight, particularly typically from about 0.2 to about 2.25% by weight and especially from about 0.25 to about 2.00% by weight.

The compositions as contemplated herein can also be conditioning agents in addition to cleaning agents. Conditioning agents suitable as contemplated herein can be used in Component II. typically
at least one cationic surfactant, typically in a total amount of from about 0.1 to about 10% by weight, more typically from about 0.2 to about 9% by weight and particularly typically from about 0.3 to about 8% by weight, each based on the weight of Component II, and
comprise at least one further surfactant selected from anionic, amphoteric, zwitterionic and/or non-ionic surfactants, typically in a total amount of from about 0.1 to about 20% by weight, more typically from about 0.2 to about 15% by weight and particularly typically from about 0.3 to about 10% by weight, in each case based on the weight of Component II.

In a typical embodiment, the inventive compositions in Component II comprises—based on the total weight of Component II. from about 15.0 to about 60% by weight, typically from about 20.0 to about 60% by weight, particularly typically from about 25.0 to about 60% by weight and in particular from about 30.0 to about 60% by weight of at least one polyhydric C₂-C₆ alcohol b).

Suitable polyhydric alcohols (b) include alditols such as mannitol, isomalt, lactitol, sorbitol and xylitol, threat, erythritol and arabitol, about 1.2-propylene glycol, about 1.3-butylene glycol, dipropylene glycol, glycerol and/or diglycerol. Typical are about 1.2-propylene glycol, about 1.3-butylene glycol, dipropylene glycol, glycerol and/or diglycerol. Glycerine is particularly typical.

These polyhydric alcohols are well tolerated by the skin and as solvents they ensure that the solid cosmetic compositions available with them are not too solid or too difficult or slow to dissolve.

In a typical embodiment, the inventive compositions in Component II comprises—based on the total weight of Component II.—from about 0.5 to about 10% by weight, typically from about 1.0 to about 10% by weight, particularly typically from about 2.0 to about 10% by weight and in particular from about 3.0 to about 10% by weight of at least one Component c).

Suitable Components c) for the purposes of the present disclosure are saturated or unsaturated, branched or unbranched C₈-C₃₀ carboxylic acids and/or their salts, typically C₁₀-C₂₂ carboxylic acids and/or their salts and in particular coco acids, lauric acid, myristic acid, palmitic acid, stearic acid, behenic acid, oleic acid and mixtures thereof and/or the salts of these acids. Especially suitable are coco acids, stearic acid and/or the salts of these acids, especially coco acids and sodium stearate.

These compounds have proved to be particularly suitable structure-giving ingredients for the purposes of the disclosure. They can be used to formulate cosmetic compositions of sufficient strength that do not melt too low.

In a typical embodiment, the inventive compositions in Component II comprises—based on the total weight of Component II.—from about 0.5 to about 17.5% by weight, typically from about 1.0 to about 15% by weight, particularly typically from about 1.5 to about 12.5% by weight and in particular from about 2.0 to about 10% by weight of at least one Component d).

Polysaccharides suitable for the present disclosure d) are usually composed of more than ten monosaccharide units. Typical polysaccharides are the starches composed of α-D-glucose units and starch degradation product s such as amylose, amylopectin and dextrins. As contemplated herein, chemically and/or thermally modified starches are particularly advantageous, e.g. hydroxypropyl starch phosphate, dihydroxypropyl distarch phosphate or the commercial products Dry Flo®. Dextranes and their derivatives, e.g. dextran sulphate, are also typical. Non-ionic cellulose derivatives such as methyl cellulose, hydroxypropyl cellulose, hydroxypropyl methyl cellulose or hydroxyethyl cellulose are also typical. Other typical examples are polysaccharides from fucose units, such as the commercial product Fucogel®.

The present disclosure also relates to a solid cosmetic composition as described above, comprising as polysaccharide d) in Component II.
i. Starch fractions from corn, potatoes, rice, wheat and/or tapioca, and/or derivatives of starches such as amylose, amylopectin, dextrin, and/or
ii. Cellulose and/or cellulose derivatives such as alkyl cellulose, carboxyalkyl cellulose, hydroxyalkyl cellulose

Particularly typically, the compositions as contemplated herein comprise in Component II. at least one polysaccharide d) from the aforementioned Group i.. In particular, solid cosmetic compositions typically comprise Component II. (based on the total weight of Component II.)
From about 1.0 to about 15% by weight of maize starch and
From about 0.1 to about 10% by weight of dextrins, typically maltodextrin

These polysaccharides have proved to be particularly suitable stabilising agents in the context of the present disclosure claimed here. Their use makes it possible to provide ready-made consumer products that retain their properties and appearance over a long period of time and under various environmental conditions.

The present disclosure further relates to a solid cosmetic composition as described above, comprising in Component II. at least one cationic polymer, typically at least one cationic polysaccharide polymer obtainable from guar, cassia and/or inulin in a proportion by weight of from about 0.01 to about 5.00% by weight of the total weight of Component II.

Cationic polymers have conditioning properties, i.e. they provide a pleasant skin or hair feel and thus offer added value. They can be used in the context of the present disclosure without significantly affecting the cleaning performance

Especially suitable:
quaternized cellulose polymers, especially polyquaternium-10, as commercially available under the names Celquat® and Polymer JR®,
hydrophobically modified cellulose derivatives, such as the cationic polymers sold under the trade name SoftCat®,
cationic alkyl polyglycosides,
cationized honey, for example the commercial product Honeyquat® 50,
cationic polysaccharide polymers from natural sources such as guar, cassia and/or inulin
polymeric dimethyldiallylammonium salts and copolymers thereof with esters and amides of acrylic acid and methacrylic acid, especially polyquaternium-6 and polyquaternium-7 The products marketed under the names Merquat®100 (poly(dimethyldiallylammonium chloride)) and Merquat®550 (dimethyldiallylammonium chloride-acrylamide copolymer) are examples of such cationic polymers,
Copolymers of vinylpyrrolidone with quaternized derivatives of dialkylaminoalkyl acrylate and methacrylate, such as vinylpyrrolidone-dimethylaminoethylmethacrylate copolymers quaternized with diethyl sulphate. Such compounds are commercially available under the names Gafquat®734 and Gafquat®755,
Vinylpyrrolidone-vinyl imidazolium methochloride copolymers, as offered under the names Luviquat® FC 370, FC 550, FC 905 and HM 552,
quaternized polyvinyl alcohol,
and the polymers known as Polyquaternium 2, Polyquaternium 17, Polyquaternium 18, Polyquaternium-24, Polyquaternium 27, Polyquaternium-32, Polyquaternium-37, Polyquaternium 74 and Polyquaternium 89

Cationic polysaccharide polymers are particularly typical in the sense of the present disclosure,

which are obtained from natural sources (in particular from guar, cassia and/or inulin)
A content of these specific polymer types in Component II. of the compositions as contemplated herein is not only beneficial for improving the care properties of skin and hair, but it was also found that polymers in combination with other cationic polymers do not cause an over conditioning effect even after regular application on hair.

Inulin is a polysaccharide belonging to the group of fructans. In addition to a terminal glucose building block, the chain comprises up to about 60 fructose monomers, each of which is linked via β-2,1-glycosidic bonds. Inulin may be obtained from the leaves, roots, fruits and/or flowers of composites and/or umbellifers, such as Jerusalem artichokes, chicory, artichokes and/or parsnips.

Cationic inulin polymers particularly suitable as contemplated herein are cationically modified by reacting hydroxyl groups of the fructose building blocks with reactive quaternary ammonium compounds. Suitable quaternary ammonium compounds are typically compounds of the following formula

N+(R¹R²R³R⁴) X⁻

in which R¹, R² and R³ are methyl or ethyl groups and R⁴ is an epoxy-R⁵— or a halohydrin group Y—CH₂—CH(OH)—R⁵—, in which R⁵ is a C₁-C₃ alkylene group, Y is a halide and X is an anion such as Cl—, Br—, I— or HSO4. Particularly suitable cationic inulin polymers b) for the purposes of the present disclosure correspond to the formula

R—O—CH₂—CH(OH)—R⁵—N⁺(R¹R²R³) X⁻,

wherein R is inulin and the other residues have the same meaning as above.

In a particularly typical embodiment, the compositions as contemplated herein in Component II. comprise cationic inulin polymers cationically modified with cationic hydroxy-C₁-C₃-alkyl-trialkylammonium groups, in particular with hydroxypropyl trimethylammonium groups. Within this version, cationic inulin polymers known and commercially available under the INCI designation Hydroxypropyltrimonium Inulin are typical.

The degree of cationic substitution of cationic inulin polymers, especially of cationic inulins known under the INCI designation Hydroxypropyltrimonium Inulin, can be varied and adjusted as required. For the use in Component II. of the cosmetic compositions as contemplated herein, it has been shown to be particularly typical when such cationically modified inulin polymers exhibit a higher degree of cationic modification (higher degree of cationic substitution), because this can ultimately result in a better care performance

In a particularly typical embodiment, the cationic inulin polymer included in Component II. of the cosmetic compositions as contemplated herein has a cationic charge density about >1.5 meq/g, more typically about >2.0 meq/g, particularly typically about >2.5 meq/g, very typically about >3.0 meq/g and especially about >3.5 meq/g. Within this version it is particularly typical if cationic inulin polymers known under the INCI designation Hydroxypropyltrimonium Inulin b) have a cationic charge density about >1.5 meq/g, more typically about >2.0 meq/g, particularly typically about >2.5 meq/g, very particularly typically about >3.0 meq/g and particularly about >3.5 meq/g.

In a further particularly typical version, the cationic inulin polymer b) included in Component II. of the cosmetic compositions as contemplated herein has an average molar mass of from about 2,000 to about 50,000 g/mol, more typically from about 2,500 to about 40,000 g/mol, particularly typically from about 3,000 to about 30,000 g/mol, very typically from about 3,500 to about 20,000 g/mol and in particular from about 4,000 to about 10,000 g/mol. Within this version it is particularly typical if cationic inulin polymers known under the INCI designation Hydroxypropyltrimonium Inulin b) have an average molar mass of from about 2,000 to about 50,000 g/mol, more typically from about 2,500 to about 40,000 g/mol, particularly typically from about 3,000 to about 30,000 g/mol, very typically from about 3,500 to about 20,000 g/mol and particularly from about 4,000 to about 10,000 g/mol.

It was found that the combination of cationic inulin polymers and specific cationic polymers available from natural sources is particularly suitable for achieving particularly good hair care effects. Typically, cationic polymers originating from natural sources are cationic polygalactomannan derivatives.

Galactomannans are polysaccharides chosen from combinations of mannose and galactose monomers in different contents. In it, the mannose units are connected to each other via β(1-4)-glycosidic bonds; the galactose units via α(1-6)-bonds. The ratio of mannose to galactose monomers varies according to the type and origin of the plant and the temperature at which it was grown. In Greek fenugreek gum, the mannose-galactose ratio is about 1:1 (corresponding to one monomer of mannose to one monomer of galactose); in guar gum about 2:1; in tara gum about 3:1; in locust bean gum about 4:1 and in cassia gum about 5:1. All galactomannans from these sources are suitable for cationic modification and use as polymers in cosmetic compositions as contemplated herein. Guar gum and/or cassia gum are particularly suitable for use in cosmetic products as contemplated herein.

Like the cationic inulin polymers, the galactomannans, typically galactomannans from the aforementioned sources, can be cationically modified by reacting the hydroxyl groups of the galactomannan polymers with reactive quaternary ammonium compounds. Suitable quaternary ammonium compounds are typically compounds of the following formula

N³⁰ (R¹R²R³R⁴) X⁻

in which R¹, R² and R³ are methyl or ethyl groups and R⁴ is an epoxy-R⁵ - or a halohydrin group Y—CH₂—CH(OH)—R⁵—, in which R⁵ is a C₁-C₃ alkylene group, Y is a halide and X is an anion such as Cl—, Br—, I— or HSO4. Particularly suitable cationic galactomannane polymers within the meaning of the present disclosure correspond to the formula

R—O—CH₂—CH(OH)—R⁵—N⁺(R¹R²R³) X⁻,

wherein R is the respective galactomannan and the other radicals have the same meaning as above.

In a particularly typical embodiment, the compositions as contemplated herein therefore comprise cationic galactomannan polymers cationically modified with cationic hydroxy-C₁-C₃-alkyl-trialkylammonium groups, in particular with hydroxypropyltrimethylammonium groups. Within this version, galactomannan polymers which have been cationically modified with cationic hydroxy-C₁-C₃-alkyl-trialkylammonium groups, in particular with hydroxypropyltrimethylammonium groups, and which are derived from guar gum and/or cassia gum are particularly typical.

In a particularly typical embodiment, the cosmetic compositions as contemplated herein comprise in Component II. as cationic polymer e) at least one cationic polysaccharide polymer e) from natural sources, typically a cationic polymer obtainable from guar, cassia and/or inulin and particularly typically one or more of the compounds known under the INCI designations guar hydroxypropyltrimonium chloride, hydroxypropyl guar hydroxypropyltrimonium chloride, hydroxypropyl trimonium inulin and/or cassia hydroxypropyltrimonium chloride. The cationic polymer known under the INCI designation Hydroxypropyl Trimonium Inulin is particularly typical.

The present disclosure further relates to a solid cosmetic composition as described above, comprising in Component II. at least one oil, fat and/or wax component, typically a naturally occurring oil, fat or wax in a proportion of from about 0.01 to about 10.00% by weight of the total weight of Component II.

These are caring substances that help to keep both the skin and hair structure healthy. The defined concentration range makes it possible to use this care effect but at the same time to exclude noticeable greasiness after application of an appropriate composition. Naturally occurring raw materials have the advantage that they grow again and can therefore be used sustainably. This aspect is also becoming increasingly important to many users.

It has been found that vegetable butters with a melting range of from about 20° C. to about 35° C. are particularly suitable for incorporation into cosmetic compositions as contemplated herein.

Accordingly, vegetable butters with a melting point in the range from 20° C. to 35° C., such as Shea butter (INCI designation), are particularly typical: Butyrospermum Parkii (Shea) Butter), Mango Butter (INCI designation: Mangifera Indica (Mango) Seed Butter), Murumuru Butter (INCI designation: Astrocaryum Murumuru Seed Butter), cocoa butter (INCI designation: Theobroma Cacao (Cocoa) Seed Butter) and/or Cupuacu Butter (INCI designation: Theobroma Grandiflorum Seed Butter).

Cupuacu butter (INCI designation) is particularly typical: Theobroma Grandiflorum Seed Butter) and/or Shea Butter (INCI designation: Butyrospermum Parkii (Shea) Butter) and especially typical is Shea Butter (INCI designation: Butyrospermum Parkii (Shea) Butter).

The at least one vegetable butter (typically Cupuacu butter and/or Shea butter; especially Shea butter) is used in Component II. of the cosmetic compositions as contemplated herein typically in a proportion by weight of from about 0.01 to about 10.00% by weight, more typically of from about 0.05 to about 5% by weight, particularly typically of from about 0.10 to about 1% by weight of the total weight of Component II.

Oils suitable as contemplated herein are typically perfume oils and/or vegetable triglyceride oils, such as coconut oil, (sweet) almond oil, walnut oil, peach kernel oil, apricot kernel oil, avocado oil, tea tree oil, soy bean oil, cotton seed oil, sesame oil, sunflower oil, Tsubaki oil, evening primrose oil, rice bran oil, palm oil, Palm kernel oil, mango kernel oil, cranberry oil, sea buckthorn oil, meadow foam herb oil, thistle oil, macadamia nut oil, grape seed oil, amaranth seed oil, argan oil, bamboo oil, olive oil, Wheat germ oil, pumpkin seed oil, mallow oil, hazelnut oil, safflower oil, corn oil, olive oil, rapeseed oil, canola oil, sasanqua oil, jojoba oil, rambutan oil, manila oil and/or quinoa oil.

Particularly typical are argan oil, jojoba oil, marula oil, macadamia nut oil, pumpkin seed oil, amaranth seed oil, quinoa oil, soy bean oil, cotton seed oil, sunflower oil, palm oil, palm kernel oil, linseed oil, almond oil, corn oil, olive oil, rapeseed oil, sesame oil, soy bean oil, thistle oil, wheat germ oil, peach kernel oil, cranberry oil, sea buckthorn oil and/or coconut oil.

Especially typical are argan oil, jojoba oil, marula oil, almond oil, olive oil, coconut oil and/or sunflower oil.

The oil(s) can be used in Component II. of the compositions as contemplated herein typically in a proportion by weight of from about 0.01 to about 10.0%, more typically from about 0.05 to about 5.0%, particularly typically from about 0.10 to about 3.0%, of the total weight of Component II.

The present disclosure further relates to a solid cosmetic composition as described above, comprising in Component I and/or II at least one bitter substance, typically a compound known under the INCI designation denatonium benzoate in a proportion by weight of from about 0.001 to about 0.01% by weight of the total weight of Component I and/or II.

A bitter substance is particularly important in cosmetics, household products etc. which are made up in such a way that their shape, colour, feel etc. appeal to small children or babies and encourage them to play, although swallowing could also occur. A bitter substance prevents this. Denatonium benzoate is an extremely strong bittering agent and is therefore particularly effective even at exceptionally low application concentrations. Furthermore, it is not associated with any known adverse effects.

In addition to the ingredients described above, the cosmetic compositions claimed in the present disclosure may be used in Component II. may also comprise at least one active substance advantageously selected from the group comprising plant extracts, humectants, protein hydrolysates, perfumes, UV filters, structurants such as maleic acid, dyes for colouring the composition, active substances such as bisabolol and/or allantoin, antioxidants, Preservatives such as sodium benzoate or salicylic acid, additional viscosity regulators such as salts (NaCl) or polymers, and pH adjusters such as α and β hydroxycarboxylic acids such as citric acid, lactic acid, malic acid, glycolic acid, and/or bases such as alkanolamines and/or sodium hydroxide).

Suitable plant extracts are extracts that can be produced from all parts of a plant. Usually these extracts are produced by extraction of the whole plant. However, in some cases it may he preferable to produce the extracts exclusively from flowers and/or leaves of the plant.

Especially suitable are extracts from Paeonia Lactiflora, Rosa Damascena Flower, Malus Domestica Fruit, Argani a Spinosa Shell Powder, Laminaria Saccharina, Cannabis Sativa, Green Tea, Oak bark, Nettle, Hamamelis, Hops, Chamomile, Burdock root, Horsetail, Hawthorn, Lime blossom, Litchi, Almond, Aloe Vera, Spruce needle, Horse chestnut, Sandalwood, juniper, coconut, mango, apricot, lime, wheat, kiwi, melon, orange, grapefruit, sage, rosemary, birch, mallow, cuckooflower, thyme, yarrow, Thyme, lemon balm, cowslip, marshmallow, ginseng, ginger root, Echinacea purpurea, Olea europea, Boerhavia diffusa roots, Foeniculum vulgaris and Apim graveolens.

The extracts of Paeonia Lactiflora, Rosa Damascena Flower, Malus Domestica Fruit, Argania Spinosa Shell Powder, Laminaria Saccharina, Cannabis Sativa, Green Tea, Nettle, Hamamelis, Chamomile, Aloe Vera, Ginseng, Echinacea purpurea, Olea europea and/or Boerhavia Diffusa roots are particularly typical for use in the compositions as contemplated herein.

Water, alcohols and mixtures thereof may be used as extraction agents for the preparation of the above plant extracts. Among the alcohols, lower alcohols such as ethanol and isopropanol, but especially polyhydric alcohols such as ethylene glycol and propylene glycol, both as the sole extracting agent and mixed with water, are typical. Plant extracts based on water/propylene glycol in a ratio of from about 1:10 to about 10:1 have proven to be particularly suitable.

The plant extracts can be used both in pure and diluted form. If they are used in diluted form, they usually comprise approximately 2-80% by weight of active substance and the extraction agent or mixture of extraction agents used in their extraction as solvent. The plant extracts can be used in Component IL of the hair treatment compositions as contemplated herein (based on the total weight of Component typically in an amount of 0.01 to 10% by weight, more typically of from about 0.05 to about 7.5% by weight and in particular of from about 0.1 to about 5% by weight.

The present disclosure further relates to a solid cosmetic composition as described above for the cleansing and/or care of human skin and/or human hair.

The present disclosure further relates to a solid cosmetic composition as described above, where
Component I. was coated with Component II., or
Component I. has been impregnated, sprayed or coated with Component II., or
Component II. has been rolled onto Component I., or
Component I. with Component II. was printed.

This offers the advantage of allowing the effects of the two different components to occur at different times. In addition, in this way it is possible to use a component on the inside which, on its own, would appear unattractive, for example because it would be too soft and attract small dust particles and the like. It may be attractive to be able to use such a component nevertheless, as this increases the degrees of freedom for the formulation. Apart from that, these cosmetic compositions appeal to the playful side of the user and can be enriching due to their novel properties.

The present disclosure further relates to a solid cosmetic composition as described above, whereby Component II. is located as a separate layer between two Components I. (sandwich arrangement).

This design offers the advantages just described to a particular extent, while at the same time being easy to manufacture.

The present disclosure further relates to a solid cosmetic composition as described above, comprising an additional layer III comprising a hair conditioning agent.

A hair conditioning agent within the meaning of the present disclosure is typically a solid hair conditioning agent which—based on the total weight of the hair conditioning agent (layer III.)—is typical
a) From about 0.1 to about 15.0% by weight of at least one cationic surfactant,
b) From about 10.0 to about 45.0% by weight of at least one polyhydric C₂-C₆ alcohol
c) From about 1.0 to about 15.0% by weight of at least one saturated or unsaturated, branched or unbranched C₈-C₃₀alcohol and/or a saturated or unsaturated, branched or unbranched C₈-C₃₀carboxylic acid and/or a salt of a saturated or unsaturated, branched or unbranched C₈-C₃₀carboxylic acid, and
d) From about 1.0 to about 20.0% by weight of at least one starch fraction, a modified starch and/or a starch derivative.

This gives the composition additional conditioning properties. These create a pleasant skin or hair feel, thus offering added value. In the context of the disclosure, this is possible without significantly impairing the cleaning performance The aforementioned three-layer structure is particularly suitable for this purpose.

The present disclosure further relates to a solid cosmetic composition in which Component I. is completely enveloped by component two. Component II. can be in the form of a powder, a liquid of a tablet or a shaped body which dissolves and foams up in contact with water.

This makes it possible to offer components that are otherwise difficult to handle, such as powders or liquids, as individual application portions, which, moreover, is accompanied by an interesting and therefore enriching feeling during application.

The present disclosure further relates to a solid cosmetic composition as previously described, wherein Component II. is in the form of a porous body which has a density in the range of from about 0.2 g/cm³ to about 1.2 g/cm³ and which dissolves and foams in contact with water.

A porous body feels interesting, which enriches the user sensorially, and also dissolves well and quickly due to the large surface, which can save time but also water.

The present disclosure further relates to a process for cleansing the human body, including the hair, in which a solid cosmetic composition is moistened with water as described above, soaked or dissolved in water, rubbed between the hands and foamed, spread over the part of the body to be cleansed and rinsed with water.

The present disclosure also relates to a method of applying the solid cosmetic composition as contemplated herein, in which the solid composition is either first mixed with water and then applied to the hair, or in which the solid composition is applied directly to the wet hair.

These procedures make the advantages described above tangible for a user and thus represent an enrichment not only for personal hygiene and care but also a sensory enrichment.

The present disclosure further relates to a use of a cleaning composition as described above for cleaning and/or care of the human body including the hair.

This use makes the advantages described above tangible for a user and thus represents an enrichment not only for personal hygiene and care but also a sensory enrichment.

As can be seen from the previously described designs and their advantages, the process and packaging aspects are important for the present disclosure. They are discussed in more detail below.

In a manufacturing process as contemplated herein (for Component II.), for example, all ingredients are placed in a heatable container, such as, on a laboratory scale, in a suitable vessel in a water bath or on a heating plate, on a production scale rather in a closed and pressurizable vessel, and are mixed and heated, in the recipes as contemplated herein, for example at about 75° C. until all ingredients are sufficiently mixed. In such a process, different temperature steps can also be run. For example, components that can be homogeneously mixed even at a relatively low temperature can be mixed first. This can happen from about 40° C. to about 50° C. It can also be advantageous to mix in certain ingredients at higher temperatures, for example from about 85° C. to about 90° C. For this purpose, a process as contemplated herein may comprise one or more steps in this temperature range. Afterwards, one or more steps can be carried out at a lower temperature again, in which further components are mixed in. Typically, the compositions as contemplated herein solidify at about 65° C., so that certain process steps, such as mixing and extruding the finished mixtures, are not reasonably possible below such a temperature level.

A mixture resulting from a process described above may also comprise a gas, including gas mixtures such as air, N₂, N₂O and/or CO₂. This can be done in a boiler, for example, from about 200-4000 kPa or by adding air, for example, using a high-speed mixer or similar equipment. A mixture thus obtained can then be discharged via an extruder. The mixture expands if it was too pre-pressurized, as discussed here, and solidifies at a normal room temperature of from about 18° C. to about 25° C., for example, additionally favoured by cooling, which is accompanied by the expansion of the included gas. Otherwise, if it was previously mixed under ambient pressure, the mixture will only cool down and solidify by assuming the ambient temperature or by additionally provided cooling.

The introduction of a gas or gas mixture into the compositions as contemplated herein (Component II.) is accompanied by various advantages. As explained at the beginning, good dissolving properties are important for solid cosmetic compositions, in particular for compositions which are also intended to be used for single application portions. In addition to the composition, the dissolution behaviour can be influenced by the method of production and packaging. For example, by introducing a gas or gas mixture, the surface area where contact with water can take place can be increased, resulting in faster dissolution, and the extremely fine bubble structure already established in the solid composition means that the foam resulting from dissolution is particularly fine and creamy, which is perceived as pleasant.

It is also important to note that cosmetic products have a lot to do with feeling, fun and emotions. Many people relax during personal hygiene and enjoy the pleasant feeling of doing something good for themselves. Especially since many people find their everyday life more and more demanding or stressful, small pleasures and playfulness are an important point at which stress can be released from a person and satisfaction can be created. Solid cosmetic preparations with incorporated gas phase, i.e. to a certain extent solid foams, feel different from conventional products, which is perceived as interesting and pleasant.

The extrusion processes described above can also be used to produce interesting shapes reminiscent of injection-moulded biscuits. Thanks to specially shaped dies on the extrusion die, a variety of shapes can be realized, for example a heart or clover shape. An extruded strand thus obtained can then be cut into pieces or slices, providing emotionally appealing small portions of the solid cosmetic compositions as contemplated herein. Similarly, it is possible to roll out an extruded strand or other form of a composition as contemplated herein and then, by punching or cutting, produce pieces that are similar to cookie cutters in terms of shaping properties.

There are further possibilities to create exciting, emotionally appealing products through a special implementation of a manufacturing process. Thus, a mixture as contemplated herein (II.) can be continuously applied to another Component (I.). A further layer of this other Component (I) can also subsequently be applied to the other side of the extrudate (II.), which is not yet covered with such a layer, so that a sandwich arrangement results, or another mixture can be applied as a third layer after an analogous manufacturing process. Another possibility is that a Mixture (II.) is added continuously or in portions to prefabricated moulds made of a further Component (I.) and then completely enveloped by an additional quantity of this further Component (I.), for example in the form of two hemispheres. The final density of the mixture can also be adjusted via the pressure.

Foamed solidified emulsions as such have already been mentioned above, but it should be noted here that the consistency of the foamed extrudate and the corresponding production facilities make it possible to create imaginative shapes, such as those familiar from meringue.

It is also possible to pour the solid cosmetic compositions as contemplated herein into a crucible, for example a glass jar. Since the strength of these compositions is in a range that allows an application portion to be taken manually, without a tool, from a crucible intended for multiple applications. If the composition has been foamed in a crucible during its manufacture, the result is a particularly interesting feeling.

It is also possible to make up solid cosmetic compositions reminiscent of a piece of paper, a foil or a wafer, which brings with it a new and pleasant feeling during application. Since the thickness of the coating is small in this type of packaging, short dissolution times can be achieved, which accommodates impatient users and does not encourage a wasteful use of water. A product packaged in this way may be placed on the market in a packaging unit in which a large number of leaves or flakes are placed in a small carton, possibly subdivided, so that a single withdrawal is possible.

After various designs and their respective advantages were explained in detail, the presentation of exemplary compositions and an exemplary manufacturing process follows.

Basic exemplary compositions of Component II. are shown in the following tables 1-4

TABLE 1

| Ingredient | Z1 | Z2 | Z3 | Z4 | Z5 | Z6 | Z7 | Z8 | Z9 |
|---|---|---|---|---|---|---|---|---|---|
| a): Surfactant, | 15-50 | 16-49 | 17.5-47.5 | 18-46 | 20-45 | 21-44 | 22.5-42.5 | 23-41 | 25-40 |
| especially an anionic | | | | | | | | | |
| surfactant | | | | | | | | | |
| b): Polyvalent C₂-C₄ | 10-60 | 12.5-60 | 15-60 | 17.5-60 | 20-60 | 22.5-60 | 25-60 | 27.5-60 | 30-60 |
| alcohol | | | | | | | | | |
| c): saturated or unsaturated, | 0.1-10 | 0.25-10 | 0.5-10 | 0.75-10 | 1.0-10 | 1.5-10 | 2.0-10 | 2.5-10 | 3.0-10 |
| branched or unbranched | | | | | | | | | |
| C₈-C₃₀ | | | | | | | | | |
| alcohol, and/or | | | | | | | | | |
| saturated or unsaturated, | | | | | | | | | |
| branched or unbranched | | | | | | | | | |
| C₈-C₃₀ | | | | | | | | | |
| carboxylic acid | | | | | | | | | |
| and/or its salt | | | | | | | | | |
| d): Polysaccharide | 0.1-20 | 0.25-19 | 0.5-17.5 | 0.75--16 | 1.0-15 | 1.25-14 | 1.5-13.5 | 1.75-12 | 2.0-10 |
| Water and, if | ad | ad | ad | ad | ad | ad | ad | ad | ad |
| necessary, other | 100 | 100 | 100 | 100 | 100 | 100 | 100 | 100 | 100 |
| auxiliary materials | | | | | | | | | |
| and additives | | | | | | | | | |

TABLE 2

| Ingredient | Z11 | Z11 | Z12 | Z13 | Z14 | Z15 | Z16 | Z17 | Z18 |
|---|---|---|---|---|---|---|---|---|---|
| Isethionate | 15-50 | 16-49 | 17.5-47.5 | 18-46 | 20-45 | 21-44 | 22.5-42.5 | 23-41 | 25-40 |
| surfactant and/or | | | | | | | | | |
| taurate surfactant | | | | | | | | | |
| 1.2-propylene | 10-60 | 12.5-60 | 15-60 | 17.5-60 | 20-60 | 22.5-60 | 25-60 | 27.5-60 | 30-60 |
| glycol, 1.3-butylene | | | | | | | | | |
| glycol, dipropylene | | | | | | | | | |
| glycol, glycerol | | | | | | | | | |
| and/or diglycerol | | | | | | | | | |
| Coco acids, lauric | 0.1-10 | 0.25-10 | 0.5-10 | 0.75-10 | 1.0-10 | 1.5-10 | 2.0-10 | 2.5-10 | 3.0-10 |
| acid, myristic acid, | | | | | | | | | |
| palmitic acid, stearic | | | | | | | | | |
| acid, behenic acid, | | | | | | | | | |
| oleic acid and/or | | | | | | | | | |
| their salts | | | | | | | | | |
| Starch fractions from | 0.1-20 | 0.25-19 | 0.5-17.5 | 0.75--16 | 1.0-15 | 1.25-14 | 1.5-13.5 | 1.75-12 | 2.0-10 |
| maize, potatoes, rice, | | | | | | | | | |
| wheat and/or tapioca | | | | | | | | | |
| and/or derivatives of | | | | | | | | | |
| starches such as | | | | | | | | | |
| amylose, amylopectin, | | | | | | | | | |
| dextrins and/or | | | | | | | | | |
| Cellulose and/or | | | | | | | | | |
| cellulose derivatives | | | | | | | | | |
| such as alkyl cellulose, | | | | | | | | | |
| carboxyalkyl cellulose, | | | | | | | | | |
| hydroxyalkyl cellulose | | | | | | | | | |
| Water and, if | ad | ad | ad | ad | ad | ad | ad | ad | ad |
| necessary, other | 100 | 100 | 100 | 100 | 100 | 100 | 100 | 100 | 100 |
| auxiliary materials | | | | | | | | | |
| and additives | | | | | | | | | |

TABLE 3

| Ingredient | Z19 | Z20 | Z21 | Z22 | Z23 | Z24 | Z25 | Z26 | Z27 |
|---|---|---|---|---|---|---|---|---|---|
| Isethionate surfactant | 10-40 | 11-37.5 | 12.5-35 | 14-34 | 15-32.5 | 16-31 | 17.5-30 | 18-27.5 | 20-25 |
| according to formula | | | | | | | | | |
| (I) | | | | | | | | | |
| Taurate surfactant | 1.0-10 | 1.5-9.5 | 2.0-9.0 | 2.25-8.5 | 2.5-8.0 | 2.75-7.5 | 3.0-7.0 | 3.0-7.0 | 3.0-7.0 |
| according to formula | | | | | | | | | |
| (II) | | | | | | | | | |
| Glycerine | 10-60 | 12.5-60 | 15-60 | 17.5-60 | 20-60 | 22.5-60 | 25-60 | 27.5-60 | 30-60 |
| Coco acids, stearic acid | 0.1-10 | 0.25-10 | 0.5-10 | 0.75-10 | 1.0-10 | 1.5-10 | 2.0-10 | 2.5-10 | 3.0-10 |
| and/or their salts | | | | | | | | | |
| Corn starch | 1.0-15 | 1.1-14 | 1.25-13.5 | 1.4-13 | 1.5-12.5 | 1.75-12 | 2.0-11.5 | 2.5-11 | 3.0-10 |
| Maltodextrin | 0.1-10 | 0.2-9.0 | 0.3-8.5 | 0.4-8.0 | 0.5-7.5 | 0.6-7.0 | 0.7-6.5 | 0.75-6.0 | 0.75-5.5 |
| Water and, if | ad | ad | ad | ad | ad | ad | ad | ad | ad |
| necessary, other | 100 | 100 | 100 | 100 | 100 | 100 | 100 | 100 | 100 |
| auxiliary materials | | | | | | | | | |
| and additives | | | | | | | | | |

TABLE 4

| Ingredient | Z28 | Z29 | Z30 | Z31 | Z32 | Z33 | Z34 |
|---|---|---|---|---|---|---|---|
| a): Surfactant, especially | 15-50 | 15-50 | 15-50 | 15-50 | 15-50 | 15-50 | 15-50 |
| an anionic surfactant | | | | | | | |
| b): polyvalent C₂-C₄ | 10-60 | 10-60 | 10-60 | 10-60 | 10-60 | 10-60 | 10-60 |
| alcohol | | | | | | | |
| c): saturated or unsaturated, | 0.1-10 | 0.1-10 | 0.1-10 | 0.1-10 | 0.1-10 | 0.1-10 | 0.1-10 |
| branched or unbranched | | | | | | | |
| C₈-C₃₀ | | | | | | | |
| alcohol, and/or | | | | | | | |
| saturated or unsaturated, | | | | | | | |
| branched or unbranched | | | | | | | |
| C₈-C₃₀ | | | | | | | |
| carboxylic acid | | | | | | | |
| and/or its salts | | | | | | | |
| d): Polysaccharide | 0.1-20 | 0.1-20 | 0.1-20 | 0.1-20 | 0.1-20 | 0.1-20 | 0.1-20 |
| e) cationic polymer | 0.01-5.0 | | | 0.01-5.0 | 0.01-5.0 | | 0.01-5.0 |
| f): Oil, fat and/or | | 0.01-5.0 | | 0.01-5.0 | | 0.01-5.0 | 0.01-5.0 |
| wax component | | | | | | | |
| g): Bitter seed | | | 0.001-0.01 | | 0.001-0.01 | 0.001-0.01 | 0.001-0.01 |
| Water and, if necessary, | ad | ad | ad | ad | ad | ad | ad |
| other auxiliary materials | 100 | 100 | 100 | 100 | 100 | 100 | 100 |
| and additives | | | | | | | |

Detailed exemplary compositions of Component II. are shown in the following table 5

TABLE 5

| | | active substances included | |
|---|---|---|---|
| Group | Ingredients | therein (INCI) | Z 36 |
| 1 | Pureact ® WS Conc | Aqua (Water), Sodium Methyl | 11.15 |
| | | Cocoyl Taurate, Coconut Acid, | |
| | | Sodium Chloride | |
| 1 | City water | Aqua (Water) | 0.25 |
| 1 | Glycerine 99.5% | Glycerine | 41 |
| 1 | Sodium stearate OP- | Sodium Stearate | 6 |
| | 100 V | | |
| 1 | Citric acid | Citric Acid | 2 |
| 2 | ISELUX ® | Sodium Lauroyl Methyl | 2 |
| | | Isethionate, Sodium Methyl | |
| | | Isethionate | |
| 2 | Maltodextrin DE15 | Maltodextrin | 1 |
| 3a | Pureact I-80P ® | Sodium Cocoyl Isethionate | 25.5 |
| 3b | Maisita 9040 ® | Zea Mays (Corn) Starch | 5 |
| 4 | Avocado oil, | Persea Gratissima (avocado) Oil, | 2 |
| | cold pressed | Ascorbyl palmitate | |
| 4 | Preservatives | Phenoxyethanol | 1 |
| 4 | Fragrance | Perfume (Fragrance) | 1 |

The exemplary procedure was carried out as follows:

The composition shown in detail in Table 5 was produced as follows: The ingredients were used in the ratio shown in Table 2 above. Pureact WS Conc was heated in a drum to about 40° C. to about 50° C. to melt and mix. After mixing until homogeneity, the other ingredients of group 1 (see table 2) were added. It was mixed again until homogeneity and then the temperature was increased to about 85° C. to about 90° C. At this temperature, the ingredients of Group 2 (see Table 2) were added and mixed in until homogeneous. This was then repeated with the ingredients of Group 3 (3a and 3b, see Table 2). The ingredients of Group 4 (see Table 2) were homogeneously mixed together and also added to the previously prepared mixture and mixed in until homogeneity was achieved. For the next steps, the temperature was no longer actively maintained at about 85° C. to about 90° C., but it was only ensured that it did not drop to about 70° C. or less. Then all ingredients of Group 5 (see Table 2) were added sequentially and mixed in until homogeneous and the ingredient of Group 6 (see Table 2) was added and quickly mixed in until homogeneous. Finally, the mixture was kept at a temperature above about 70° C. for filling or packaging.

This was followed by the introduction of a gas selected from air, N₂, N₂O and/or CO₂ at a pressure of about 2 to about 40 bar or alternatively the introduction of air with a high speed mixer, extrusion of the gassed mixture from a desired shaping orifice or into a desired mould and solidification/cooling of the extrudate in the desired shape and removal of the extrudate from the mould or cutting and portioning of the extrudate. The cosmetic compositions obtained had densities ranging from about 0.2 g/cm³ to about 1.2 g/cm³.

Up to this point, the product of the process corresponded to a Component II described above, and it was further developed as a separate layer between two layers of Component I. (comprising circular leaflets (wafers) made from about >90% by weight potato starch and about <10% by weight % Water; based on the total weight of Component I.) temporarily stored (sandwich arrangement) in order to obtain a cosmetic composition as contemplated herein.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A solid cosmetic composition comprising at least two separate Components I. and II., wherein
said at least one Component I comprises at least one of:
a. starch fractions from corn, potatoes, rice, wheat and/or tapioca,
b. cereal flour and/or
c. sugar chosen from glucose, sucrose, fructose, maltose, and
said at least one Component II comprises:
a. from about 15.0 to about 50.0% by weight of at least one surfactant,
b. from about 10.0 to about 60.0% by weight of at least one polyhydric C₂-C₆ alcohol, p2 c. from about 0.1 to about 10.0% by weight of at least one saturated or unsaturated, branched or unbranched C₈-C₃₀-alcohol and/or a saturated or unsaturated alcohol, branched or unbranched C₈-C₃₀ carboxylic acid and/or a salt of a saturated or unsaturated, branched or unbranched C₈-C₃₀ carboxylic acid, and
d. from about 0.1 to about 20.0% by weight of at least one polysaccharide chosen from
starch fractions, modified starches, derivatives of starches, and
cellulose and/or cellulose derivatives,
wherein the quantities are based on the total weight of said at least one Component II.

2. The solid cosmetic composition according to claim 1, wherein Component I. comprises—based on its total weight—from about 75 to about 100% by weight of one or more ingredient(s) a. to c.

3. The solid cosmetic composition according to claim 1, comprising (based on the total weight of Component II.)
a.i) from about 10.0 to about 40% by weight of at least one anionic isethionate surfactant and
b.ii) from about 1.0 to about 10% by weight of at least one anionic taurate surfactant.

4. The solid cosmetic composition according to claim 2, comprising (based on the total weight of Component II.)
a.i) from about 10.0 to about 40% by weight of at least one anionic isethionate surfactant and
b.ii) from about 1.0 to about 10% by weight of at least one anionic taurate surfactant.

5. The solid cosmetic composition according to claim 1, wherein polyhydric alcohol b) in Component II. comprises 1.2-propylene glycol, 1.3-butylene glycol, dipropylene glycol, glycerol and/or diglycerol.

6. The solid cosmetic composition according to claim 2, wherein polyhydric alcohol b) in Component II. comprises 1.2-propylene glycol, 1.3-butylene glycol, dipropylene glycol, glycerol and/or diglycerol.

7. The solid cosmetic composition according to claim 1, wherein Component c) in Component II. comprises saturated or unsaturated, branched or unbranched C₈-C₃₀-carboxylic acids and/or their salts.

8. The solid cosmetic composition according to claim 2, wherein Component c) in Component II. comprises saturated or unsaturated, branched or unbranched C₈-C₃₀-carboxylic acids and/or their salts.

9. The solid cosmetic composition according to claim 1, wherein Component c) in Component II. comprises C₁₀-C₂₂-carboxylic acids and/or their salts.

10. The solid cosmetic composition according to claim 2, wherein Component c) in Component II. comprises C₁₀-C₂₂-carboxylic acids and/or their salts.

11. The solid cosmetic composition according to claim 1, wherein Component c) in Component II. comprises a coco acid, lauric acid, myristic acid, palmitic acid, stearic acid, behenic acid, oleic acid and mixtures thereof and/or the salts of these acids.

12. The solid cosmetic composition according to claim 1, wherein polysaccharide d) in Component II. is chosen from at least one of:
i. amylose, amylopectin, and dextrin; and/or
ii. alkyl cellulose, carboxyalkyl cellulose, and hydroxyalkyl cellulose.

13. The solid cosmetic composition according to claim 1, wherein
Component II. is coated with Component I., or
Component II. is impregnated, sprayed or coated with Component I., or
Component II. is rolled onto Component I., or
Component I. is printed onto Component II.

14. The solid cosmetic composition according to claim 1, wherein Component II. is disposed as a separate layer sandwiched between a first and a second Component I.

15. The solid cosmetic composition according to claim 1 comprising an additional layer III which comprises a hair conditioning agent.

16. The solid cosmetic composition according to claim 1, wherein Component II. is in the form of a porous body which has a density in the range from about 0.2 g/cm³ to about 1.2 g/cm³ and which dissolves and foams in contact with water.

17. The solid cosmetic composition according to claim 1, wherein Component II. is present in the form of a powder, a liquid of a tablet or a shaped body, and wherein Component II. is completely enveloped by Component I.

18. The solid cosmetic composition according to claim 1,
wherein Component I. comprises—based on its total weight—from about 75 to about 100% by weight of one or more ingredient(s) a. to c.;
wherein the solid composition further comprises (based on the total weight of Component II.)
a.i) from about 10.0 to about 40% by weight of at least one anionic isethionate surfactant and
b.ii) from about 1.0 to about 10% by weight of at least one anionic taurate surfactant;
wherein polyhydric alcohol b) in Component II. comprises 1.2-propylene glycol, 1.3-butylene glycol, dipropylene glycol, glycerol and/or diglycerol;
wherein Component c) in Component II. comprises saturated or unsaturated, branched or unbranched Cs-Cm-carboxylic acids and/or their salts; and
wherein polysaccharide d) in Component II. is chosen from at least one of amylose, amylopectin, dextrin, alkyl cellulose, carboxyalkyl cellulose, and/or hydroxyalkyl cellulose.

19. The solid cosmetic composition according to claim 1,
wherein Component I. comprises—based on its total weight—from about 75 to about 100% by weight of one or more ingredient(s) a. to c.;
wherein the solid composition further comprises (based on the total weight of Component II.)
a.i) from about 10.0 to about 40% by weight of at least one anionic isethionate surfactant and
b.ii) from about 1.0 to about 10% by weight of at least one anionic taurate surfactant;
wherein polyhydric alcohol b) in Component II. comprises C₁₀-C₂₂-carboxylic acids and/or their salts;
wherein Component c) in Component II. comprises saturated or unsaturated, branched or unbranched C₈-C₃₀-carboxylic acids and/or their salts; and
wherein polysaccharide d) in Component II. is chosen from at least one of amylose, amylopectin, dextrin, alkyl cellulose, carboxyalkyl cellulose, and/or hydroxyalkyl cellulose.

20. The solid cosmetic composition according to claim 1,
wherein Component I. comprises—based on its total weight—from about 75 to about 100% by weight of one or more ingredient(s) a. to c.;
wherein the solid composition further comprises (based on the total weight of Component II.)
a.i) from about 10.0 to about 40% by weight of at least one anionic isethionate surfactant and p1 b.ii) from about 1.0 to about 10% by weight of at least one anionic taurate surfactant;
wherein polyhydric alcohol b) in Component II. comprises 1.2-propylene glycol, 1.3-butylene glycol, dipropylene glycol, glycerol and/or diglycerol;
wherein Component c) in Component II. comprises a coco acid, lauric acid, myristic acid, palmitic acid, stearic acid, behenic acid, oleic acid and mixtures thereof and/or the salts of these acids; and
wherein polysaccharide d) in Component II. is chosen from at least one of amylose, amylopectin, dextrin, alkyl cellulose, carboxyalkyl cellulose, and/or hydroxyalkyl cellulose.
